(19) United States
(12) Patent Application Publication
(10) Pub. No.: US 2014/0171706 A1
(43) Pub. Date: Jun. 19, 2014

(54) METHODS AND APPARATUSES FOR FORMING LOW-AROMATIC HIGH-OCTANE PRODUCT STREAMS

(71) Applicant: UOP LLC, Des Plaines, IL (US)

(72) Inventor: Bryan K. Glover, Algonquin, IL (US)

(73) Assignee: UOP LLC, Des Plaines, IL (US)

(21) Appl. No.: 13/715,843

(22) Filed: Dec. 14, 2012

(51) Int. Cl. C07C 5/22

(52) U.S. Cl. CPC C07C 5/22
USPC 585/317; 585/350; 422/630; 422/187

(57) ABSTRACT

Methods and apparatuses for forming low-aromatic high-octane product streams and methods for processing hydrocarbons are provided. In an embodiment, a method for forming a low-aromatic high-octane product stream includes deisohexanizing or deisoheptanizing a hydrocarbon stream and forming a cycloalkane-rich stream. Further, the method includes isomerizing the cycloalkane-rich stream at equilibrium conditions favoring formation of cyclopentanes over cyclohexanes.

### TECHNICAL FIELD

The technical field generally relates to methods and apparatuses for forming low-aromatic high-octane product streams from cycloalkane-rich streams, and more particularly relates to methods and apparatuses for increasing the ratio of cyclopentanes to cyclohexanes in cycloalkane-rich streams.

### BACKGROUND

Gasoline having high octane numbers, e.g., Research Octane Number (RON), is required for modern gasoline engines. Formerly it was common to accomplish octane number improvement by the use of various lead-containing additives. As lead was phased out of gasoline for environmental reasons, octane ratings were maintained through use of other aromatic and low vapor pressure hydrocarbons. However, due to harmful biological and environmental effects of aromatics, standards have been enacted to regulate their content in fuels such as gasoline. For example, certain standards may limit gasoline to concentrations of no more than 35 wt % aromatics. At the same time, fuel standards continue to require high octane numbers for gasoline. Therefore, it has become increasingly necessary to rearrange the structure of the C5 and C6 hydrocarbons used in gasoline blending in order to obtain high octane levels.

Common hydrocarbon processing schemes for light naphtha isomerization use a post-isomerization deisohexanizer to separate streams based on boiling points. Typically the bottom stream from a deisohexanizer is cycloalkane (naphthene)-rich, has a low octane number, and can impart a significant negative impact on the overall octane of the combined isomerate product. This is especially true when processing hydrocarbon feeds having a high cyclic C6 content.

One process to increase the octane number of a deisohexanizer bottom stream delivers the bottom stream to a catalytic reforming unit. While the high cycloalkane content of the bottom stream makes it an ideal reforming unit feed, catalytic reforming will produce additional aromatics in the naphtha complex. As discussed above, modern gasoline processing seeks to minimize the concentration of aromatics.

Accordingly, it is desirable to provide novel methods and apparatuses for upgrading the octane numbers of cycloalkane-rich streams. It is also desirable to provide methods and apparatuses for processing deisohexanizer or deisoheptanizer bottom streams to increase the ratio of methylcyclopentane to cyclohexane therein without increasing aromatic concentrations. Also, it is desirable to provide such methods and apparatuses that operate economically. Furthermore, other desirable features and characteristics will become apparent from the subsequent detailed description and the appended claims, taken in conjunction with the accompanying drawings and the foregoing technical field and background.

### BRIEF SUMMARY

Methods and apparatuses for forming low-aromatic high-octane product streams and methods for processing hydrocarbons are provided. In one exemplary embodiment, a method for forming a low-aromatic high-octane product stream includes deisohexanizing or deisoheptanizing a hydrocarbon stream and forming a cycloalkane-rich stream. Further, the method includes isomerizing the cycloalkane-rich stream at equilibrium conditions favoring formation of cyclopentanes over cyclohexanes.

In another embodiment, a method for processing hydrocarbons is provided. The method for processing hydrocarbons includes providing a cycloalkane-rich stream. Further, the method includes converting a first portion of cycloalkanes in the cycloalkane-rich stream to cyclopentanes and converting a second portion of cycloalkanes in the cycloalkane-rich stream to cyclohexanes. The method also includes forming a product stream from the cyclopentanes and the cyclohexanes, wherein the ratio of cyclopentanes to cyclohexanes is at least 6:5.

In another embodiment, an apparatus for upgrading the octane number of a cycloalkane-rich stream is provided. The apparatus includes a deisohexanizer zone configured to form the cycloalkane-rich stream as a bottom stream from a hydrocarbon stream. Further, the apparatus includes an upgrading zone configured to isomerize the cycloalkane-rich stream at equilibrium conditions favoring formation of cyclopentanes over cyclohexanes.

### BRIEF DESCRIPTION OF THE DRAWINGS

FIG. 1 is a schematic diagram of an apparatus and method for upgrading the octane numbers of cycloalkane-rich streams in accordance with an embodiment.

### DETAILED DESCRIPTION

The following detailed description is merely exemplary in nature and is not intended to limit the methods or apparatuses for upgrading the octane numbers of cycloalkane-rich streams. Furthermore, there is no intention to be bound by any theory presented in the preceding background or the following detailed description.

Methods and apparatuses for processing hydrocarbons, and more particularly, for upgrading octane numbers of cycloalkane streams, such as deisohexanizer or deisoheptanizer bottom streams, are provided herein. The methods and apparatuses achieve hydrocarbon product streams with higher octane numbers (RON) without raising levels of aromatics therein. More specifically, cycloalkane-rich streams, such as deisohexanizer or deisoheptanizer bottom streams, are isomerized to upgrade their octane numbers by preferential isomerization of cycloalkanes to methylcyclopentane over cyclohexanes and over aromatics.

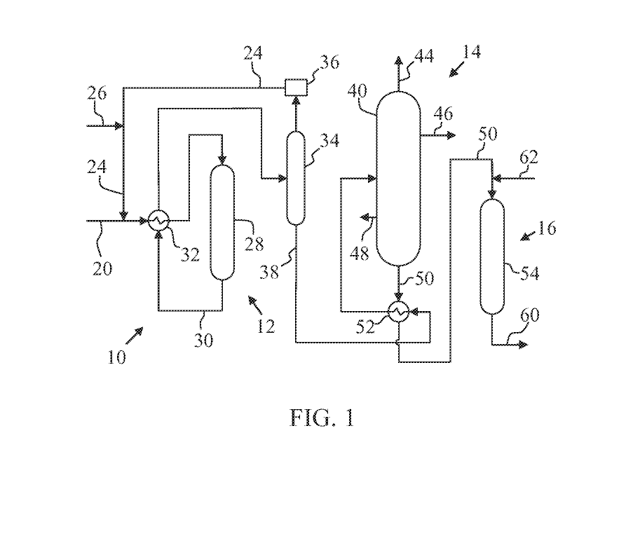

In FIG. 1, an exemplary embodiment of an apparatus and method for upgrading an octane number of a deisohexanizer or deisoheptanizer bottom stream is described. Reference to the specific arrangement in FIG. 1 is not meant to limit the apparatus and method to the details disclosed therein. Furthermore, FIG. 1 is a schematic illustration and does not show a number of details for the process arrangement such as pumps, compressors, valves, and recycle lines that are well-known to those skilled in the art.

FIG. 1 shows an apparatus 10 with three primary operating zones: an isomerization zone 12, a deisohexanizer or deisoheptanizer zone 14, and an upgrading zone 16. As shown, a feedstock stream 20 is introduced to the isomerization zone 12. The feedstocks that can be used herein include hydrocarbon fractions rich in C4 to C7 normal paraffins and cyclics. As used herein, the term “rich” does not have a minimum required amount other than greater than zero. One category of feedstocks are normal and cyclic paraffin streams having from 4 to 7 carbon atoms or a mixture of such paraffins. Other useful feedstocks include light natural gasoline, light straight run naphtha, gas oil condensate, light raffinates, light reformate, light hydrocarbons, field butanes, and straight run distillates having distillation end points of about 77° C. (170° F.) and containing substantial quantities of C4-C6 paraffins. The feedstock may also contain low concentrations of unsaturated hydrocarbons. In a particular embodiment, the feedstock includes a high level of C6 cyclics, or a high level of C7 cyclics, such as more than about 5 wt %, or more than about 10 wt % of the selected cyclics.

Hydrogen 24 is admixed with the feedstock stream 20 in an amount that will provide a hydrogen-to-hydrocarbon ratio equal to or less than 0.05 in the isomerization effluent when operating without hydrogen recycle. If necessary, make-up gas or additional hydrogen 26 can be provided to the hydrogen 24 as shown. For some catalyst systems, such as platinum on chlorided alumina, the hydrogen-to-hydrocarbon (H2/HC)) ratio of 0.05 or less at the effluent has been found to provide sufficient excess hydrogen for operation of the process. Zeolitic or sulfated zirconia systems require higher H2/HC ratios. Although no net hydrogen is consumed in the isomerization reaction, the isomerization zone will have a net consumption of hydrogen, often referred to as the stoichiometric hydrogen requirement, which is associated with a number of side reactions that occur. These side reactions include cracking and disproportionation. Other reactors that will also consume hydrogen include olefin and aromatics saturation. For feedstocks having a low level of unsaturates, satisfying the stoichiometric hydrogen requirements demands a hydrogen-to-hydrocarbon molar ratio for the outlet stream of between about 0.01 to about 10.0. Hydrogen in excess of the stoichiometric amounts for the side reactions is maintained in the isomerization zone to provide good stability and conversion by compensating for variations in feedstock compositions that alter the stoichiometric hydrogen requirements.

When the hydrogen-to-hydrocarbon ratio exceeds 0.10, it is not economically desirable to operate the isomerization process without the recycle of hydrogen to the isomerization zone. As the quantity of hydrogen leaving the product recovery section increases, additional amounts of C4 and other product hydrocarbons are taken by the fuel gas stream from the product recovery section. The value of the lost product or the additional expense associated with recovery facilities to prevent the loss of product do not justify operating the process without recycle at hydrogen-to-hydrocarbon ratios above 0.10. However, when employing hydrogen recycle, hydrogen is admixed with the feed in an amount that will provide a hydrogen-to-hydrocarbon ratio equal to from about 0.01 to about 10.0 in the effluent from the isomerization zone

Hydrogen 24 may be added to the feed mixture in any manner that provides the necessary control for the addition of small hydrogen quantities. Metering and monitoring devices for this purpose are well known by those skilled in the art. A control valve may be used to meter the addition of hydrogen 24 to the feedstock stream 20. The hydrogen concentration in the effluent may be monitored by a hydrogen monitor and the control valve setting position may be adjusted to maintain the desired hydrogen concentration. The hydrogen concentration at the effluent is calculated on the basis of total effluent flow rates.

As shown, the isomerization zone 12 includes a single isomerization reactor 28 that contains an isomerization catalyst. The isomerization zone 12 is not restricted to that particular design but can consist of any type of isomerization zone that takes a stream of C5-C6 straight-chain hydrocarbons or a mixture of straight-chain, branched-chain and cyclic hydrocarbons and converts straight-chain hydrocarbons in the feed mixture to branched-chain hydrocarbons and branched hydrocarbons to more highly branched hydrocarbons thereby producing an effluent having branched-chain and straight-chain hydrocarbons. A two-reactor system with a first stage reactor and a second stage reactor in the reaction zone is a common alternative embodiment. For a two reactor system, the catalyst used is distributed between the two reactors in any reasonable distribution. It is not necessary that the reaction be carried out in two reactors but the use of two reactors confers several benefits on the process. The use of two reactors and specialized valving allows partial replacement of the catalyst system without taking the isomerization unit off stream. For the short periods of time during which replacement of catalyst may be necessary, the entire flow of reactants may be processed through only one reaction vessel while catalyst is replaced in the other. The use of two reaction zones also aids in maintaining lower catalyst temperatures. This is accomplished by having any exothermic reaction such as hydrogenation of unsaturates performed in the first vessel with the rest of the reaction carried out in a final reactor stage at more favorable temperature conditions. For example, the relatively cold hydrogen and hydrocarbon feed mixtures may be passed through a cold feed exchanger that heats the incoming feed against the effluent from the final reactor. The feed from the cold feed exchanger is carried to the hot feed exchanger where the feed is heated against the effluent carried from the first reactor. The partially heated feed from hot feed exchanger may be carried through an inlet exchanger that supplies any additional heat requirements for the feed and then into a first reactor. Effluent from the first reactor may be carried to the second reactor after passage through an exchanger to provide inter-stage cooling. The isomerization zone effluent may be carried from second reactor through the cold feed exchanger as previously described and into the separation facilities.

The feedstock stream 20 and hydrogen 24 are contacted in the isomerization reactor 28 with an isomerization catalyst. The catalyst composites that can be used in the isomerization reactor 28 include traditional isomerization catalysts including chlorided platinum alumina, crystalline aluminosilicates or zeolites, and other solid strong acid catalysts such as sulfated zirconia and modified sulfated zirconia. Suitable catalyst compositions of this type will exhibit selective and substantial isomerization activity under the operating conditions of the process.

As a class, the crystalline aluminosilicate or crystalline zeolite catalysts comprise crystalline zeolitic molecular sieves having an apparent pore diameter large enough to adsorb neopentane. A silica alumina molar ratio SiO₂:Al₂O₃ of greater than 3, less than 60, and preferably between 15 and 50 is desirable. In one form, the zeolite will contain an equivalent percentage of alkali metal cations and will have those AlO₄-tetrahedra that are not associated with alkali metal cations—including those not associated with any metal cations or those associated with divalent or other polyvalent metal cations. Usually the molecular sieve is a mordenite molecular sieve, which is essentially in the acid form or is converted to the acid form.

A composition of zeolitic catalyst for use herein comprises a Group VIII noble metal, a hydrogen form crystalline aluminosilicate, and a refractory inorganic oxide with the catalyst composition having a surface area of at least about 580 m²/g. Significant improvements in isomerization performance are realized when the surface area of the catalytic composite is at or above 580 m²/g. A Group VIII metal is incorporated into the catalytic composite to supply a hydrogenation/dehydrogenation function and a particular Group VIII noble metal is platinum. The Group VIII noble metal is present in an amount from about 0.01 to about 5% by weight of the composite and preferably in an amount of at least about 0.15% by weight but not over about 0.35% by weight. The zeolitic catalytic composite may also contain a catalytically effective amount of a promoter metal such as tin, lead, germanium, cobalt, nickel, iron, tungsten, chromium, molybdenum, bismuth, indium, gallium, cadmium, zinc, uranium, copper, silver, gold, tantalum, or one or more of rare earth metals and mixtures thereof The hydrogen-formed silica alumina has either a three-dimensional or channel pore structure crystal lattice framework. The three-dimensional aluminosilicates include both synthetic and naturally occurring silica aluminas such as faujasites, which include X-type, Y-type, ultrastable-Y, and the like. L-type, omega-type, and mordenite are examples of the channel pore structure crystalline aluminosilicates. Mordenite, in either naturally occurring or synthetic form is one embodiment, particularly with a silica to alumina ratio of at least about 16:1. The hydrogen form aluminosilicate may be present in an amount within the range of about 50 to about 99.5 wt %, preferably within the range of about 75 to about 95 wt %, and a refractory inorganic oxide may be present in an amount within the range of from about 25 to about 50 wt %.

Another suitable isomerization catalyst is a solid strong acid catalyst that comprises a sulfated support of an oxide or hydroxide of a Group IVB (IUPAC 4) metal, preferably zirconium oxide or hydroxide, at least a first component that is a lanthanide element or yttrium component, and at least a second component being a platinum-group metal component. The catalyst optionally contains an inorganic-oxide binder, especially alumina

The support material of the solid strong acid catalyst comprises an oxide or hydroxide of a Group IVB (IUPAC 4). In one embodiment the Group IVB element is zirconium or titanium. Sulfate is composited on the support material. A component of a lanthanide-series element is incorporated into the composite by any suitable means. The lanthanide series element component may be selected from the group consisting of lanthanum, cerium, praseodymium, neodymium, promethium, samarium, europium, gadolinium, terbium, dysprosium, holmium, erbium, thulium, ytterbium and lutetium. Suitable amounts of the lanthanide series element component are in the range of about 0.01 to about 10 mass % on an elemental basis, of the catalyst. A platinum-group metal component is added to the catalytic composite by any means known in the art to effect the catalyst, e.g., by impregnation. The platinum-group metal component may be selected from the group consisting of platinum, palladium, ruthenium, rhodium, iridium, or osmium. Amounts in the range of from about 0.01 to about 2 wt % platinum-group metal component, on an elemental basis are suitable.

Optionally, the catalyst is bound with a refractory inorganic oxide. The binder, when employed, usually comprises from about 0.1 to 50 mass %, preferably from about 5 to 20 mass %, of the finished catalyst. The support, sulfate, metal components and optional binder may be composited in any order effective to prepare a catalyst useful for the isomerization of hydrocarbons. Examples of suitable atomic ratios of lanthanide or yttrium to platinum-group metal for this catalyst are at least about 1:1; for example about 2:1 or greater; such as about 5:1 or greater. The catalyst may optionally further include a third component of iron, cobalt, nickel, rhenium or mixtures thereof. For example, iron may be present in amounts ranging from about 0.1 to about 5 wt % on an elemental basis. In an exemplary embodiment, the solid strong acid isomerization catalyst is sulfated zirconia or a modified sulfated zirconia.

Another class of suitable isomerization catalysts for use herein includes the chlorided platinum alumina catalysts. The aluminum is preferably an anhydrous gamma-alumina with a high degree of purity. The catalyst may also contain other platinum group metals. The term “platinum group metals” refers to noble metals excluding silver and gold that are selected from the group consisting of platinum, palladium, germanium, ruthenium, rhodium, osmium, and iridium. These metals demonstrate differences in activity and selectivity such that platinum has now been found to be the most suitable for this process. The catalyst will contain from about 0.1 to about 0.25 wt % of the platinum. Other platinum group metals may be present in a concentration of from about 0.1 to about 0.25 wt %. The platinum component may exist within the final catalytic composite as an oxide or halide or as an elemental metal. The presence of the platinum component in its reduced state has been found most suitable for this process. The chloride component termed in the art “a combined chloride” is present in an amount from about 2 to about 10 wt % based upon the dry support material. The use of chloride in amounts greater than about 5 wt % has been found to be the most beneficial for this process. The inorganic oxide preferably comprises alumina and more preferably gamma-alumina, eta-alumina, and mixtures thereof.

There are a variety of ways for preparing the catalytic composite and incorporating the platinum metal and the chloride therein. One method prepares the catalyst by impregnating the carrier material through contact with an aqueous solution of a water-soluble decomposable compound of the platinum group metal. For best results, the impregnation is carried out by dipping the carrier material in a solution of chloroplatinic acid. Additional solutions that may be used include ammonium chloroplatinate, bromoplatinic acid or platinum dichloride. Use of the platinum chloride compound serves the dual function of incorporating the platinum component and at least a minor quantity of the chloride into the catalyst. Additional amounts of halogen must be incorporated into the catalyst by the addition or formation of aluminum chloride to or on the platinum-aluminum catalyst base. An alternate method of increasing the halogen concentration in the final catalyst composite is to use an aluminum hydrosol to form the aluminum carrier material such that the carrier material also contains at least a portion of the chloride. Halogen may also be added to the carrier material by contacting the calcined carrier material with an aqueous solution of the halogen acid such as hydrogen chloride.

It is generally known that high chlorided platinum-alumina catalysts of this type are highly sensitive to sulfur and oxygen-containing compounds. Therefore, the use of such catalysts requires that the feedstock be relatively free of such compounds. A sulfur concentration no greater than about 0.5 ppm is generally required for use of high chloride platinum-alumina catalysts. The presence of sulfur in the feedstock serves to temporarily deactivate the catalyst by platinum poisoning. Activity of the catalyst may be restored by hot hydrogen stripping of sulfur from the catalyst composite or by lowering the sulfur concentration in the incoming feed to below about 0.5 ppm so that the hydrocarbon will desorb the sulfur that has been adsorbed on the catalyst. Water can act to permanently deactivate the catalyst by removing high activity chloride from the catalyst and replacing it with inactive aluminum hydroxide. Therefore, water, as well as oxygenates, in particular C1-C5 oxygenates, that can decompose to form water, can only be tolerated in very low concentrations. In general, this requires a limitation of oxygenates in the feed to about 0.1 ppm or less. The feedstock may be treated by any method that will remove water and sulfur compounds. Sulfur may be removed from the feedstock stream by hydrotreating. A variety of commercial dryers are available to remove water from the feed components. Adsorption processes for the removal of sulfur and water from hydrocarbon streams are also well known to those skilled in the art.

Operating conditions within the isomerization zone are selected to maximize the production of isoalkane product from the feed components. Temperatures within the reaction zone will usually range from about 40° to about 235° C. (100° to 455° F.). Lower reaction temperatures usually favor equilibrium mixtures of isoalkanes versus normal alkanes. Lower temperatures are particularly useful in processing feeds composed of C5 and C6 alkanes where the lower temperatures favor equilibrium mixtures having the highest concentration of the most branched isoalkanes. When the feed mixture is primarily C5 and C6 alkanes, temperatures in the range of from about 60° to about 160° C. are suitable. Thus, when the feed mixture contains significant portions of C4-C6 alkanes, the most suitable operating temperatures are in the range of from about 145° to about stream 385° C. The isomerization zone may be maintained over a wide range of pressures. Pressure conditions in the isomerization of C4-C6 paraffins range from about 700 KPaa to about 7000 KPaa. In other embodiments pressures for this process are in the range of from about 20 barsg to 50 barsg. The feed rate to the reaction zone can also vary over a wide range. These conditions include liquid hourly space velocities ranging from about 0.5 to about 12 hr⁻¹ however, with some embodiments having space velocities between about 1 and about 6 hr⁻¹.

In FIG. 1, the isomerization effluent 30 exits the isomerization zone 12 and is processed to separate desired isomerized products from hydrogen, light ends, lower octane isomerized products, and cycloalkanes (naphthenes) plus heavy hydrocarbons having 7 or more carbon atoms. First, the isomerization effluent 30 may be heat exchanged with the feedstock stream 20 at heat exchanger 32 before being passed to a separator 34. The isomerization effluent 30 enters separator 34, which divides the isomerization effluent 30 into a heavy stream 38 comprising C4 and heavier hydrocarbons, and the overhead hydrogen stream 24 that is made up of hydrogen and lighter hydrocarbons including C3 and lighter boiling compounds. Certain embodiment will not utilize a separator, for example, an isomerization zone 12 using a platinum on chlorided alumina catalyst running 0.05 H2/HC will not use a separator.

Conditions for the operation of the separator 34 include pressures ranging from about 689 to about 4137 KPaa (100 to 600 psia). Specific embodiments utilize pressures from about 1379 to about 3447 KPaa (200 to about 500 psia). Suitable designs for rectification columns and separator vessels suitable for use as the separator 34 are well known to those skilled in the art. The hydrogen stream 24 is recycled using recycle compressor 36 to combine with feedstock stream 20. Additional hydrogen 26, if necessary, may be added to recycle hydrogen stream 24.

After separation, the heavy stream 38 may be heat exchanged with other streams, as shown in FIG. 1, before being passed to the deisohexanizer or deisoheptanizer zone 14. The heavy stream 38 is directed to a deisohexanizer or deisoheptanizer column 40. In an exemplary embodiment, the deisohexanizer or deisoheptanizer column 40 runs at a pressure of from about 138 to about 1379 KPaa (20 to 200 psia), preferably about 345 to about 689 KPaa (50 to 100 psia). Deisohexanizer or deisoheptanizer column 40 serves a variety of purposes. For example, it may provide an overhead stream 44 that contains light gases and butane. In an exemplary deisohexanizer operation, the cut point for the overhead stream 44 is above the boiling point of 2,3-dimethylbutane and below the boiling point of butane. 2,3-dimethylbutane has the higher octane of the dimethylbutane isomers and butane is considered to be too light for gasoline blending. As a result, a good split between overhead stream 44 and upper side-cut stream 46 is desired to maximize octane and minimize the amount of light material in the upper side-cut stream 46. The amount of butane taken off from the deisohexanizer column 40 will vary depending upon the amount of butane entering the process. In other embodiments, a stabilizer column will be used upstream of the deisohexanizer to remove light ends, and the deisohexanizer will only have a liquid overhead product. When processing C7s, the deisoheptanizer column 40 produces a bottoms steam rich in C7 cyclics with an overhead of trimethyl butanes and dimethyl pentanes and a lower side draw would of n-heptane and methyl hexanes. While the remaining description focuses on C6 streams in a deisohexanizer, the subject matter is not so limited and may be considered application to a deisoheptanizer for processing C7 streams.

In FIG. 1, the overhead stream 44 may be recovered for further processing or for use as fuel gas. For example, the overhead stream 44 may be separated to form an offgas containing primarily light gasses and butane and a recycle stream containing C5⁺ (C5 and greater) hydrocarbons for recycle to the deisohexanizer column 40 (not shown).

In FIG. 1, deisohexanizer column 40 provides the upper side-cut stream 46 with a high concentration of normal pentane, methylbutane and dimethylbutanes. The deisohexanizer column 40 also provides a lower side-cut C6 recycle stream 48 that comprises some normal hexane and monomethylpentanes. Upper-side cut stream 46 may also contain butane and light gases. The relatively higher octane hydrocarbons, normal pentane, methylbutane and dimethylbutanes, in upper-side cut stream 46 can be recovered from the deisohexanizer column 40 in any manner Preferably the upper side-cut stream 46 exits as a side-cut from a single deisohexanizer column 40. In an exemplary operation of deisohexanizer column 40, the cut point for the upper side-cut stream 46 is above the boiling point of 2,3-dimethylbutane and below the boiling point of 2-methylpentane. 2,3-dimethylbutane has the higher octane of the dimethylbutane isomers and 2-methylpentane has a relatively low octane number, lower than 3-methylpentane. Therefore, a good split between the lower side-cut stream 48 and the upper side-cut stream 46 is desired to maximize octane. Since only a narrow boiling point difference separates 2,3-dimethylbutane and 2-methylpentane, the deisohexanizer is designed to maximize this separation. The upper side-cut stream 46 containing normal pentane, methylbutane and dimethylbutanes may be further processed to remove light ends including butane and light gases that may be combined with deisohexanizer column overhead stream 44. The most desired isomerized products, normal pentane, methylbutane and dimethylbutanes may be removed as an isomerate product stream collected or used in gasoline blending.

The relatively lower octane hydrocarbons, including normal hexane, can be recovered from the deisohexanizer column 40 in any manner Lower side-cut stream 48 may be provided as a C6 recycle stream that exits from the single deisohexanizer column 40. The cut point for lower side-cut stream 48 in deisohexanizer column 40 may be set to recycle some normal hexane to the isomerization zone 12. For example, deisohexanizer column 40 may operate with a cut point set at above the boiling point of cyclohexane.

Heavier hydrocarbons are withdrawn from the deisohexanizer column 40 as a deisohexanizer bottom stream 50. A cut point between the lower side-cut stream 48 and bottom stream 50 may introduce substantial portions of any cyclohexane into the bottom stream 50. Further, the bottom stream 50 will generally contain other C6 cycloalkanes as wells as any C7 cycloalkanes. Where a full boiling range naphtha is used as the feed to the process, the heavy hydrocarbon feed may also comprise a C7+ naphtha.

While deisohexanizer column 40 is illustrated as including two side-cuts, alternative designs may be utilized. For example, the deisohexanizer column 40 could include a single side-cut or even no side-cuts. For the purposes described herein, it is sufficient that the deisohexanizer column 40 produce a bottom stream 50 containing cycloalkanes.

As shown, the bottom stream 50 is passed to the upgrading zone 16. The bottom stream 50 may be heat exchanged with the heavy stream 38 at heat exchanger 52. Then, the bottom stream 50 is directed to an isomerization reactor 54. As shown, the upgrading zone 16 includes a single isomerization reactor 54 that contains an isomerization catalyst. The upgrading zone 16 is not restricted to that particular design but can consist of any type of isomerization scheme that takes a stream of cycloalkanes and converts them, or a portion of them, to higher octane cycloalkanes thereby producing an effluent having a higher octane number than the bottom stream 50. A two-reactor system with a first stage reactor and a second stage reactor in the upgrading zone would be an alternative embodiment. For a two reactor system, the catalyst used is distributed between the two reactors in any reasonable distribution. It is not necessary that the reaction be carried out in two reactors but the use of two reactors confers several benefits on the process as described above in reference to isomerization reactor 28.

The goal of the upgrading process is to maximize the production of methylcyclopentane over the production of cyclohexane. Because the octane number (RON) of methylcyclopentane is about 96 while the octane number of cyclohexane is 84 (RON), maximizing the ratio of methylcyclopentane to cyclohexane optimizes the octane number (RON) of the isomerized bottom stream, i.e., the product stream 60. At low temperatures favored for paraffin isomerization, production of the lower octane cyclohexane is favored. At high temperatures, such as above about 150° C., for example above about 180° C., such as above about 200° C., production of the higher octane methylcyclopentane is favored. In an exemplary embodiment, isomerization at about 205° C. shifts the isomerization equilibrium to favor methylcyclopentane sufficiently to raise the octane number of the product stream 60 by five as compared to the pre-isomerization bottom stream 50.

The isomerization reactor 54 holds an isomerization catalyst such as a chlorided alumina or similar catalyst as described above. Another exemplary isomerization catalyst is a sulfated zirconia, or similar catalyst as described above. In certain embodiment, a low acidity catalyst is used. The selection of catalyst is based on the goal of limiting cracking while allowing the cycloalkane isomerization process to proceed at the highest temperature possible to maximize the methylcyclopentane to cyclohexane ratio.

In FIG. 1, the isomerization process in the upgrading zone 16 involves a once-through hydrogen scheme, though such a scheme is not required. As shown, hydrogen 62 is admixed with the bottom stream 50 in an amount that will provide a hydrogen-to-hydrocarbon ratio sufficient to favor formation of methylcyclopentane over cyclohexane. In an exemplary embodiment, the product stream 60 is formed with a ratio of cyclopentanes to cyclohexanes of at least about 6:5, for example at least about 3:2, such as at least about 2:1.

As described herein, an apparatus and method for upgrading an octane number of a cycloalkane-rich stream have been provided. In exemplary embodiments, an apparatus and method have been described for increasing a cyclopentane to cyclohexane ratio in a deisohexanizer bottom stream. The apparatus and method described above are particularly well-suited for the formation of a product stream that has both a low aromatic level and a high octane number for gasoline blending.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the claimed subject matter in any way. Rather, the foregoing detailed description will provide those skilled in the art with a convenient road map for implementing an exemplary embodiment or embodiments. It being understood that various changes may be made in the function and arrangement of elements described in an exemplary embodiment without departing from the scope set forth in the appended claims.

1. A method for forming low-aromatic high-octane product streams comprising:
isomerizing a feedstock stream with a C6 cyclic content to form an isomerization effluent;
separating, from the isomerization effluent, a heavy stream comprising C4 and heavier hydrocarbons and a stream comprising hydrogen and C3 and lighter boiling hydrocarbons;
deisohexanizing or deisoheptanizing the heavy stream and forming an overhead stream comprising light gases and butane, an upper sidecut stream comprising normal pentane, methylbutane, and dimethylbutanes, a lower sidecut stream comprising normal hexane and monomethylpentanes, and a cycloalkane-rich stream;
admixing hydrogen with the cycloalkane-rich stream to form a second isomerization feed consisting essentially of hydrogen and the cycloalkane-rich stream; and
isomerizing the second isomerization feed at equilibrium conditions favoring formation of cyclopentanes over cyclohexanes, wherein isomerizing the second isomerization feed occurs at a temperature of at least about 200° C.

2. The method of claim 1 wherein isomerizing the second isomerization feed comprises isomerizing second isomerization feed at conditions favoring formation of methylcyclopentanes over cyclohexanes.

3-5. (canceled)

6. The method of claim 1 wherein isomerizing the second isomerization feed comprises isomerizing the second isomerization feed over a metal-containing catalyst with acidity.

7. The method of claim 1 wherein isomerizing the second isomerization feed comprises isomerizing the second isomerization feed over a chloride alumina catalyst.

8. The method of claim 1 wherein isomerizing the second isomerization feed comprises isomerizing the second isomerization feed over a sulfated zirconia catalyst.

9. (canceled)

10. The method of claim 1 wherein isomerizing the second isomerization feed at conditions favoring formation of cyclopentanes over cyclohexanes forms a product stream with an octane number (RON) at least about 5 greater than the octane number (RON) of the cycloalkane-rich stream.

11. The method of claim 1 wherein the step of isomerizing the second isomerization feed comprises:
providing the second isomerization feed;
converting a first portion of cycloalkanes in the second isomerization feed to cyclopentanes;
converting a second portion of cycloalkanes in the second isomerization feed to cyclohexanes; and
forming a product stream from the cyclopentanes and the cyclohexanes, wherein the ratio of cyclopentanes to cyclohexanes is at least about 6:5.

12. The method of claim 11 wherein converting the first portion and converting the second portion comprises isomerizing the second isomerization feed at conditions favoring formation of cyclopentanes over cyclohexanes.

13. The method of claim 11 wherein converting the first portion and converting the second portion comprises isomerizing the second isomerization feed ream at conditions favoring formation of methylcyclopentanes over cyclohexanes.

14. (canceled)

15. The method of claim 1 wherein deisohexanizing or deisoheptanizing the heavy stream comprises deisohexanizing the heavy stream.

16. The method of claim 1 wherein deisohexanizing or deisoheptanizing the heavy stream comprises deisoheptanizing the heavy stream.

17. (canceled)

18. The method of claim 11 wherein forming a product stream comprises forming a product stream from the cyclopentanes and the cyclohexanes, wherein the ratio of cyclopentanes to cyclohexanes is at least about 2:1.

19-20. (canceled)
